(19) United States
(12) Patent Application Publication
(10) Pub. No.: US 2010/0239517 A1
(43) Pub. Date: Sep. 23, 2010

(54) NOVEL CONJUGATED PROTEINS AND PEPTIDES

(76) Inventors: Stephen Brocchini, Welling Garden City (GB); Penny Bryant, Sidcup (GB); Yuehua Cong, North Finchley (GB); Ji-Won Choi, London (GB); Antony Godwin, London (GB); Keith Powell, London (GB)

Correspondence Address: NIXON & VANDERHYE, PC, 901 NORTH GLEBE ROAD, 11TH FLOOR, ARLINGTON, VA, 22203, US

(21) Appl. No.: 12/682,057

(22) PCT Filed: Oct. 8, 2008
(86) PCT No.: PCT/GB2008/003409

§ 371 Date: Apr. 8, 2010

(30) Foreign Application Priority Data
Oct. 9, 2007 (EP) 07253992.7
Jul. 21, 2008 (GB) 0813339.9

(51) Int. Cl. A61K 38/21; C07K 16/00; C07K 14/56; C07K 14/47; A61K 38/17; A61K 39/44; A61P 29/00

(52) U.S. Cl. 424/78.3; 530/391.1; 530/351; 424/85.7; 530/350; 514/21.2; 424/179.1; 525/54.1; 530/410

(57) ABSTRACT

The invention provides a novel process for conjugating a polymer, especially PEG, to a protein or peptide, which comprises reacting a polymeric conjugation reagent with a protein or peptide containing a polyhistidine tag under conditions such that conjugation occurs via said polyhistidine tag. The resulting conjugates are novel. The invention further relates to novel conjugates of the general formula (I) in which one of X and X′ represents a polymer, and the other represents a hydrogen atom; each Q independently represents a linking group; W represents an electron-withdrawing moiety or a moiety preparable by reduction of an electron-withdrawing moiety; or, if X′ represents a polymer, X-Q-W— together may represent an electron withdrawing group; and in addition, if X represents a polymer, X′ and electron withdrawing group W together with the interjacent atoms may form a ring; Z represents a protein or a peptide linked to A and B via respective histidine residues; A is a C₁₋₅ alkylene or alkenylene chain; and B is a bond or a C₁₋₄ alkylene or alkenylene chain.

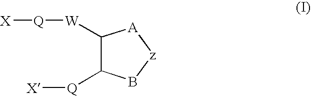

This invention relates to novel conjugated proteins and peptides, and their preparation.

Many therapeutically active molecules, for example proteins, do not possess the properties required to achieve efficacy in clinical medical use. For example, many native proteins do not make good medicines because upon administration to patients there are several inherent drawbacks that include: (1) proteins are digested by many endo- and exo-peptidases present in blood or tissue; (2) many proteins are immunogenic to some extent; and (3) proteins can be rapidly excreted by kidney ultrafiltration and by endocytosis. Some molecules which might find utility as active therapeutic agents in medicines are systemically toxic or lack optimal bioavailability and pharmacokinetics. When proteins clear from the blood circulation quickly they typically have to be administered to the patient frequently. Frequent administration further increases the risk of toxicity, especially immunologically derived toxicities.

Water soluble, synthetic polymers, particularly polyalkylene glycols, are widely used to conjugate therapeutically active molecules such as proteins. These therapeutic conjugates have been shown to alter pharmacokinetics favourably by prolonging circulation time and decreasing clearance rates, decreasing systemic toxicity, and in several cases, displaying increased clinical efficacy. The process of covalently conjugating polyethylene glycol, PEG, to proteins is commonly known as “PEGylation”.

It is important for optimised efficacy and to ensure dose to dose consistency that the number of conjugated polymer molecules per protein is the same for each molecule, and that each polymer molecule is specifically covalently conjugated to the same amino acid residue in each protein molecule. Non-specific conjugation at sites along a protein molecule results in a distribution of conjugation products and, frequently, unconjugated protein, to give a complex mixture that is difficult and expensive to purify.

WO 2005/007197 describes a series of novel conjugation reagents. These can be used to react with nucleophilic groups in a biological molecule, for example a protein, to produce a protein-polymer conjugate. These reagents find particular utility for their ability to conjugate with both sulphur atoms derived from a disulfide bond in a protein to give novel thioether conjugates.

Immobilized-metal affinity chromatography (IMAC) is a standard technique for the purification of proteins and peptides. In this technique, a polyhistidine tag, often referred to as a “his-tag” (a short chain of contiguous histidine residues, typically 5 or 6 residues, not present in the native protein) is attached by synthetic methods to a protein, generally to one of the termini of the amino acid chain. The resulting protein or peptide is said to be “histidine tagged”. Polyhistidine tags bind strongly to metals such as nickel and cobalt, via at least two of the histidine residues. In IMAC, polyhistidine tagged proteins are passed over a nickel- or cobalt-containing column. The polyhistidine tag binds strongly to the column, thus enabling the tagged protein to be separated from mixtures. Polyhistidine tags are widely used, being attached to a wide range of proteins and peptides to enable them or products derived therefrom to be separated from mixtures at a future date.

Some untagged proteins contain histidine residues in close proximity to each other, and such proteins may also bind to nickel and cobalt IMAC columns, although usually less strongly than polyhistidine tagged proteins.

We have now found an improved method of conjugating a polymer, especially PEG, to proteins and peptides. In this method, the polymer is conjugated to a polyhistidine tag. It is believed that, surprisingly, reagents capable of binding to a histidine residue will preferentially react at the polyhistidine tag, rather than at a single histidine residue present in the native protein. The resulting conjugates are novel. The reagents of WO 2005/007197 act as extremely efficient and stable conjugating agents for proteins and peptides containing polyhistidine tags, as well as for proteins and peptides containing similar histidine structures in the native protein to produce novel conjugates.

Accordingly, the present invention provides a method of conjugating a polymer to a protein or peptide containing a polyhistidine tag, which comprises reacting a polymeric conjugation reagent with said protein or peptide under conditions such that conjugation occurs via said polyhistidine tag. The invention further provides a method of conjugating a polymer to a protein or peptide, which comprises introducing a polyhistidine tag into said protein or peptide, and subsequently reacting said protein or peptide with a polymeric conjugation reagent under conditions such that conjugation occurs via said polyhistidine tag.

The polymer conjugates of the present invention are novel, and the invention therefore also provides a compound comprising a polymer conjugated to a protein or peptide, said conjugation being via a polyhistidine tag, per se. Such compounds may be represents by the general formula:

X-Q′-(his)ᵤ-Z (I)

in which X represents a polymer; Q′ represents a linking group; (his)ᵤ represents a polyhistidine tag containing u histidine units; and Z represents a protein or peptide linked through said polyhistidine tag.

Any polymeric conjugation reagent capable of bonding to a polyhistidine tag may be used in the process of the invention. Such reagents include the bifunctional reagents of WO 2005/007197, which as well as binding to polyhistidine tags, have also been found to be capable of binding to two histidine residues present in a native protein or peptide (i.e. not being present in a polyhistidine tag) where such residues are located sufficiently close together in the native structure to enable conjugation of the bifunctional reagent to both histidine residues.

Accordingly, the present invention further provides a compound of the general formula

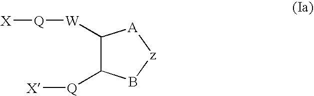

in which one of X and X′ represents a polymer, and the other represents a hydrogen atom;
each Q independently represents a linking group;
W represents an electron-withdrawing moiety or a moiety preparable by reduction of an electron-withdrawing moiety; or, if X′ represents a polymer, X-Q-W— together may represent an electron withdrawing group; and in addition, if X represents a polymer, X′ and electron withdrawing group W together with the interjacent atoms may form a ring;
Z represents a protein or a peptide linked to A and B via respective histidine residues;
A is a C₁₋₅ alkylene or alkenylene chain; and
B is a bond or a C₁₋₄ alkylene or alkenylene chain.

A further group of novel compounds according to the invention is represented by the general formula:

X-[Q-W—(CH═CH)ᵥ—(CH₂)₂-Z]_{v′} (Ib)

in which X represents a polymer; Q represents a linking group; W represents an electron-withdrawing group or a moiety preparable by reduction of an electron-withdrawing moiety; v represents 0 or an integer of from 1 to 4; v′ represent an integer of from 1 to 8; and Z represents a protein or a peptide linked via a polyhistidine tag. In these compounds, v′ preferably represents an integer from 1 to 6, preferably 1 to 4, for example 1. Preferably v is 0.

Other reagents which may be used in the process of the present invention include reagents that are capable of acylation or alkylation. Such reagents may be mono or multifunctional and include for example PEG carbonates (e.g. PEG-p-nitrophenyl carbonate, PEG-succinimidyl carbonate, PEG-benzotriazolyl carbonate), PEG carboxylates and PEG esters (e.g. PEG-succinimidyl ester and PEG-p-nitrophenyl ester and their derivatives), PEG aldehyde, PEG-tresyl or -tosyl, PEG-dichlorotriazine or -chlorotriazine, PEG vinyl sulfone, PEG maleimide and PEG-iodoacetamide; and corresponding reagents containing polymers other than PEG.

Z in the novel conjugates of the present invention may be derived from a protein or a peptide. Throughout this specification, the term “protein” will be used for convenience, and except where the context requires otherwise, references to “protein” should be understood to be references to “protein or peptide”.

The protein forming part of the novel conjugate of the formula Ia must contain at least two histidine residues. These two residues may be present in the native protein, and if so, the two residues must be located sufficiently close together in the native structure to enable conjugation to the groups A and B. This may occur when two histidine residues are adjacent to each other in the protein chain, or they may be close together as a result of the folding of the protein. Such proteins generally bind to IMAC columns. For example, the prokaryotic organism Escherichia coli has one protein known as YODA where binding to IMAC columns can occur. Preferably, however, the two histidine residues form part of a polyhistidine tag, i.e. a histidine chain, not found in the native protein or peptide, which has been attached to the protein or peptide by suitable means, for example by chemical means, by post-translational labelling with a polyhistidine tag or a moiety containing a polyhistidine tag, or via protein engineering by inserting histidine sequences in fusion with the target protein, for example by the use of a gene having a short coding sequence which codes for a polyhistidine tag of the desired length.

Polyhistidine tags may contain any desired number of histidine residues, for example up to about 12 residues. They must contain at least 2 residues; preferably they contain at least 3 residues, especially from 4 to 10 residues, especially from 5 to 8 residues, for example 5 or 6 residues. They may contain only histidine residues, or they may also contain one or more spacer residues or sequences in addition to histidine residues.

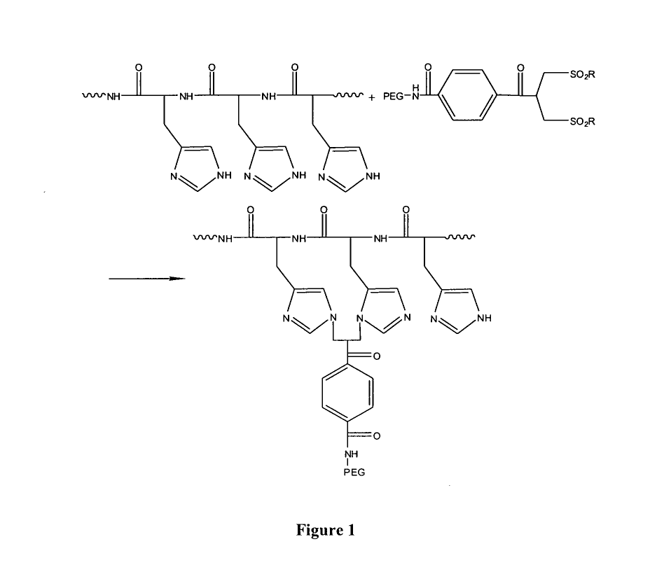

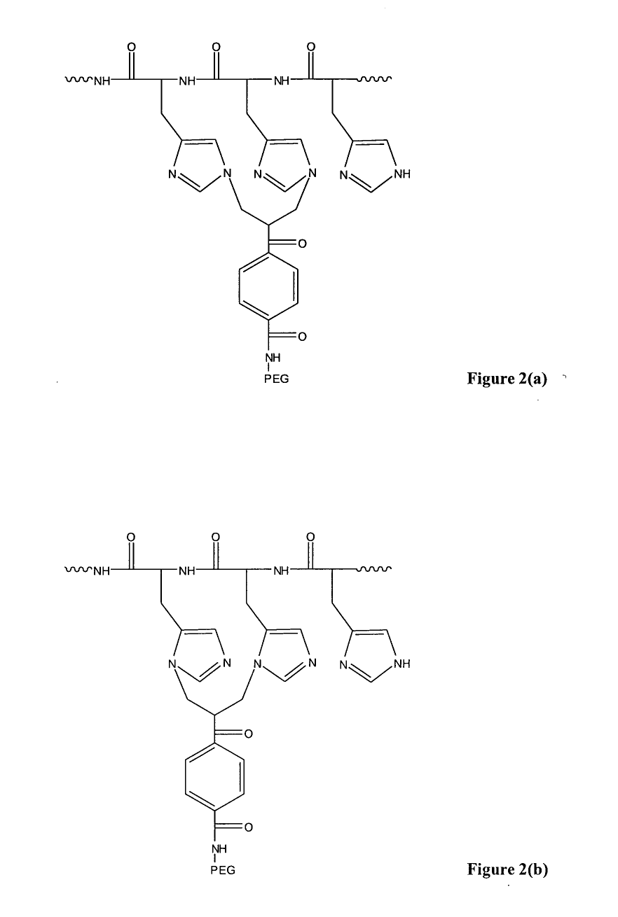

The exact nature of the binding present in the conjugates of the present invention is not currently known. The preparation of one possible conjugate, illustrated using a specific reagent in which each SO₂R represents a leaving group, and in which R preferably represents an alkyl, aryl, alkaryl or aralkyl group, especially a tolyl group, is shown in FIG. 1 of the accompanying drawings. FIGS. 2(a) and 2(b) show alternative conjugates preparable by the process of FIG. 1. It is assumed in FIGS. 1 and 2 that bonding is to adjacent histidine residues, but bonding to spaced apart histidine residues, providing that the spacing is not too large to prevent formation of the conjugate, cannot be ruled out.

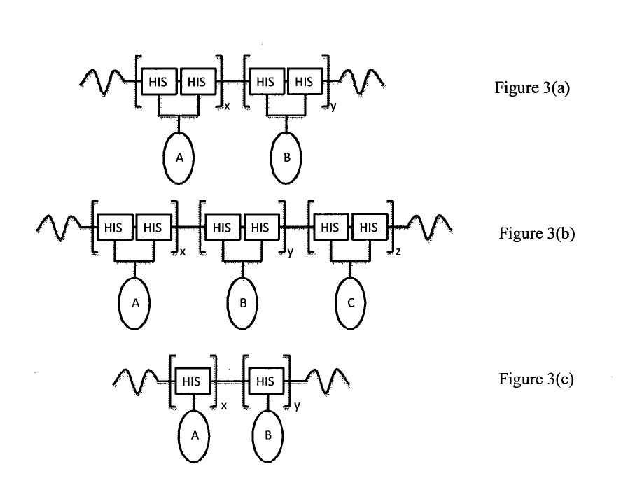

In addition to carrying a polyhistidine tag, the protein may be derivatised or functionalised if desired. In particular, prior to conjugation, the native protein may have been reacted with various blocking groups to protect sensitive groups thereon; or it may have been previously conjugated with one or more polymers or other molecules, either using the process of this invention or using an alternative process. Further, the conjugates of the invention may have one or more additional polymers (including proteins) or other molecules conjugated to the protein after conjugation according to the invention. Novel conjugates according to the present invention in which the protein is conjugated to one or two additional polymers are shown in FIGS. 3(a), 3(b) and 3(c) respectively of the accompanying drawings. In these Figures, polymers together with their linking groups are shown schematically as A, B and C, while x, y and z represent the total number of polymers A, B and C conjugated in any combination to the protein via histidine residues. Of course, conjugation of one or more polymers via residues other than histidine is also possible. As well as being conjugated to one or more additional polymers (including proteins), the protein may be conjugated to one or more molecules selected, for example, from small molecules, for example therapeutics or diagnostics; sialic acid; sugars; and starches. B and C in FIG. 3 may therefore represent such molecules. In addition, FIG. 3 shows A, B and C located adjacent each other, but this is merely schematic, and they may be spaced apart from each other.

Many proteins supplied commercially contain polyhistidine tags, either because the protein has been previously purified by way of IMAC, or to aid future purification of the protein itself or products derived from it. If it is desired to conjugate the protein to a polymer, conjugation to the polyhistidine tag provides an extremely convenient route, previously not envisaged. Conjugated products, specifically PEGylated products, can be obtained with a high degree of consistency. Use of the conjugating reagents of

WO 2005/007197, resulting in conjugation to two histidine residues, results in a macrocycle, leading to a particularly stable and selective conjugate, generally obtained in high yield.

Because the polyhistidine tag is generally attached to the surface of the protein, generally at one end of the protein chain, and can be positioned at any desired site in the protein, the biological activity of the protein is largely maintained following the process for introducing a polyhistidine tag, and also following the subsequent process of site-specific conjugation at the polyhistidine tag to a polymer. For these reasons, the present invention can be used to form conjugates of proteins which have previously proved intractable to traditional conjugation processes.

Provided that a polyhistidine tag contains a sufficient number of histidine residues, a conjugate of the present invention may still be purified using IMAC. For this to be effective, at least two, preferably more, histidine residues in the polyhistidine tag need to remain unconjugated and available for binding to the nickel in the IMAC column. The reduction in the number of free histidine residues post conjugation allows for selectivity in binding to the IMAC column between unconjugated peptide or protein and the conjugated derivative. Where multiple conjugation occurs to the same biological molecule, IMAC allows for separation of the multi-conjugated derivatives.

A polymer in a conjugate of the invention (e.g. X or X′) may for example be a polyalkylene glycol, a polyvinylpyrrolidone, a polyacrylate, for example polyacryloyl morpholine, a polymethacrylate, a polyoxazoline, a polyvinylalcohol, a polyacrylamide or polymethacrylamide, for example polycarboxymethacrylamide or a polyacrylate or polymethacrylate with phosphatidyl choline pendent groups, such as 2-methacryloyloxyethyl phosphorylcholine, or an HPMA copolymer. Additionally, the polymer may be one which is susceptible to enzymatic or hydrolytic degradation. Such polymers, for example, include polyesters, polyacetals, poly(ortho esters), polycarbonates, poly(imino carbonates), and polyamides, such as poly(amino acids). The polymer may be a homopolymer, random copolymer or a structurally defined copolymer such as a block copolymer. For example it may be a block copolymer derived from two or more alkylene oxides, or from poly(alkylene oxide) and either a polyester, polyacetal, poly(ortho ester), or a poly(amino acid). Polyfunctional polymers that may be used include copolymers of divinylether-maleic anhydride and styrene-maleic anhydride.

Naturally occurring polymers may also be used, for example polysaccharides such as chitin, dextran, dextrin, chitosan, starch, cellulose, glycogen, poly(sialylic acid) and derivatives thereof. A protein may be used as the polymer. This allows conjugation of one protein, for example an antibody or antibody fragment, to a second protein, for example an enzyme or other active protein. Also, if a peptide containing a catalytic sequence is used, for example an O-glycan acceptor site for glycosyltransferase, it allows the incorporation of a substrate or a target for subsequent enzymatic reaction. Polymers such as polyglutamic acid may also be used, as may hybrid polymers derived from natural monomers such as saccharides or amino acids and synthetic monomers such as ethylene oxide or methacrylic acid.

If the polymer is a polyalkylene glycol, this is preferably one containing C₂ and/or C₃ units, and is especially a polyethylene glycol. A polymer, particularly a polyalkylene glycol, may contain a single linear chain, or it may have branched morphology and can be composed of many chains either small or large. The so-called Pluronics are an important class of PEG block copolymers. These are derived from ethylene oxide and propylene oxide blocks. Substituted polyalkylene glycols, for example methoxypolyethylene glycol, may be used. In a preferred embodiment of the invention, a single-chain polyethylene glycol is initiated by a suitable group, for example an alkoxy, e.g. methoxy, aryloxy, carboxy or hydroxyl group, and is connected at the other end of the chain to a linking group Q or Q′ as discussed below.

The polymer may optionally be derivatised or functionalised in any desired way. Reactive groups may be linked at the polymer terminus or end group, or along the polymer chain through pendent linkers; in such case, the polymer is for example a polyacrylamide, polymethacrylamide, polyacrylate, polymethacrylate, or a maleic anhydride copolymer. Multimeric conjugates that contain more than one biological molecule, can result in synergistic and additive benefits. If desired, the polymer may be coupled to a solid support using conventional methods.

The optimum molecular weight of the polymer will of course depend upon the intended application. Preferably, the number average molecular weight is in the range of from 250 g/mol, for example 500 g/mole, to around 75,000 g/mole. When the compound of the general formula I is intended to leave the circulation and penetrate tissue, for example for use in the treatment of inflammation caused by malignancy, infection or autoimmune disease, or by trauma, it may be advantageous to use a lower molecular weight polymer in the range 2000-30,000 g/mole. For applications where the compound of the general formula I is intended to remain in circulation it may be advantageous to use a higher molecular weight polymer, for example in the range of 20,000-75,000 g/mole.

The polymer to be used should be selected so the conjugate is soluble in the solvent medium for its intended use. For biological applications, particularly for diagnostic applications and therapeutic applications for clinical therapeutic administration to a mammal, the conjugate will be soluble in aqueous media. Many proteins such as enzymes have utility in industry, for example to catalyze chemical reactions. For conjugates intended for use in such applications, it may be necessary that the conjugate be soluble in either or both aqueous and organic media. The polymer should of course not unduly impair the intended function of the protein.

Preferably the polymer is a synthetic polymer, and preferably it is a water-soluble polymer. The use of a water-soluble polyethylene glycol is particularly preferred for many applications.

The polymer is suitably covalently linked to the polyhistidine tag via a linking group. A linking group Q′ in the formula I may include a group W (i.e. Q′ may be equivalent to -Q-W— of formula Ia). A linking group Q′ or Q may for example be a direct bond, an alkylene group (preferably a C₁₋₁₀ alkylene group), or an optionally-substituted aryl or heteroaryl group, any of which may be terminated or interrupted by one or more oxygen atoms, sulphur atoms, —NR groups (in which R has the meaning given below), keto groups, —O—CO— groups and/or —CO—O— groups. Suitable aryl groups include phenyl and naphthyl groups, while suitable heteroaryl groups include pyridine, pyrrole, furan, pyran, imidazole, pyrazole, oxazole, pyridazine, pyrimidine and purine. The linkage to the polymer may be by way of a hydrolytically labile bond, or by a non-labile bond.

Substituents which may be present on an optionally substituted aryl or heteroaryl group include for example one or more of the same or different substituents selected from —CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺H₃, —N⁺HR₂, —N⁺H₂R, halogen, for example fluorine or chlorine, —C≡CR, —C═CR₂ and —C═CHR, in which each R or R′ independently represents a hydrogen atom or an alkyl (preferably C₁₋₆ alkyl) or an aryl (preferably phenyl) group. The presence of electron withdrawing substituents is especially preferred.

Preferably the group Q which links to a hydrogen atom in a compound of the formula Ia is an alkylene group or a direct bond. Most preferably, X in formula Ia is a polymer, and X′-Q- is H—.

W may for example represent a keto or aldehyde group CO, an ester group —O—CO— or a sulphone group —SO₂—, or a group obtained by reduction of such a group, e.g. a CH.OH group, an ether group CH.OR, an ester group CH.O.C(O)R, an amine group CH.NH₂, CH.NHR or CH.NR₂, or an amide CH.NHC(O)R or CH.N(C(O)R)₂. If X-Q-W— together represent an electron withdrawing group, this group may for example be a cyano group.

The precise conditions used in the conjugation process of the invention will of course depend upon which conjugation reagent is being used, and which protein or peptide is being conjugated. Such conditions are within the common knowledge of the skilled man. For example, the pH for the reaction will generally be between pH 4 to pH 10, typically between about 6 and about 8.5, for example about 6.5 to 8.0, preferably about 7.0-7.5. The concentration of the protein or peptide in the reaction mixture will generally be above 0.20 mg/ml and typically above 0.4 mg/ml, for example 0.5 mg/ml to 1.5 mg/ml. Where the process uses the difunctional reagents of WO 2005/007197, the process comprises reacting either (i) a compound of the general formula

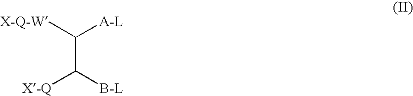

in which one of X and X′ represents a polymer and the other represents a hydrogen atom;
Q represents a linking group;
W′ represents an electron-withdrawing group, for example a keto group, an ester group —O—CO— or a sulphone group —SO₂—; or, if X′ represents a polymer, X-Q-W′ together may represent an electron withdrawing group;
A represents a C₁₋₅ alkylene or alkenylene chain;
B represents a bond or a C₁₋₄ alkylene or alkenylene chain; and
each L independently represents a leaving group; or (ii) a compound of the general formula

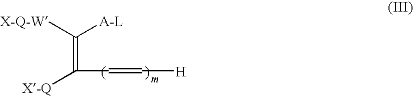

in which X, X′, Q, W′, A and L have the meanings given for the general formula II, and in addition if X represents a polymer, X′ and electron-withdrawing group W′ together with the interjacent atoms may form a ring, and m represents an integer 1 to 4; with a protein or a peptide containing at least two histidine residues.

The conjugation reagents II and III are chemically equivalent to each other, and are described in WO 2005/007197. They are characterised by having a cross-functionalised, latently cross-conjugated, bis-alkylating moiety that is selective for two nucleophiles. The reaction is carried out under conditions such that the reagent binds to two histidine residues in the protein to be conjugated. It is surprising that high selectivity to two histidine residues may be obtained. Other conjugation methods tend to produce mixtures of products; for example, it has previously been found difficult to bind selectively to histidine residues rather than to lysine residues.

The or each leaving group L may for example represent —SR, —SO₂R, —OSO₂R, —N⁺R₃, —N⁺HR₂, —N⁺H₂R, halogen, or —OØ, in which R has the meaning given above, and Ø represents a substituted aryl, especially phenyl, group, containing at least one electron withdrawing substituent, for example

—CN, —NO₂, —CO₂R, —COH, —CH₂OH, —COR, —OR, —OCOR, —OCO₂R, —SR, —SOR, —SO₂R, —NHCOR, —NRCOR, —NHCO₂R, —NR′CO₂R, —NO, —NHOH, —NR′OH, —C═N—NHCOR, —C═N—NR′COR, —N⁺R₃, —N⁺HR₂, —N⁺H₂R, halogen, especially chlorine or, especially, fluorine, —C≡CR, —C═CR₂ and —C═CHR, in which R and R′ have the meanings given above.

Typical structures in which W′ and X′ together form a ring include

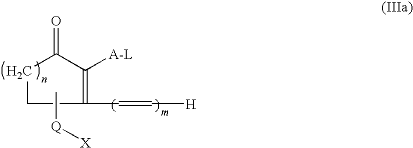

in which n is an integer from 1 to 4, and

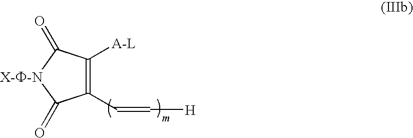

In one preferred embodiment the process according to the invention uses a reagent of the general formula:

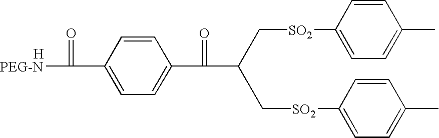

to produce a novel conjugate of the general formula:

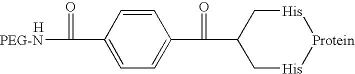

A key feature of this embodiment of the process of this invention is that an α-methylene leaving group and a double bond are cross-conjugated with an electron withdrawing function that serves as a Michael activating moiety. If the leaving group is prone to elimination in the cross-functional reagent rather than to direct displacement and the electron-withdrawing group is a suitable activating moiety for the Michael reaction then sequential intramolecular bis-alkylation can occur by consecutive Michael and retro-Michael reactions. The leaving moiety serves to mask a latent conjugated double bond that is not exposed until after the first alkylation has occurred and bis-alkylation results from sequential and interactive Michael and retro-Michael reactions as described in J. Am. Chem. Soc. 1979, 101, 3098-3110 and J. Am. Chem. Soc. 1988, 110, 5211-5212). The electron withdrawing group and the leaving group are optimally selected so bis-alkylation can occur by sequential Michael and retro-Michael reactions. It is also possible to prepare cross-functional alkylating agents with additional multiple bonds conjugated to the double bond or between the leaving group and the electron withdrawing group as described in J. Am. Chem. Soc. 1988, 110, 5211-5212.

Some examples of novel conjugates according to the invention include the following:

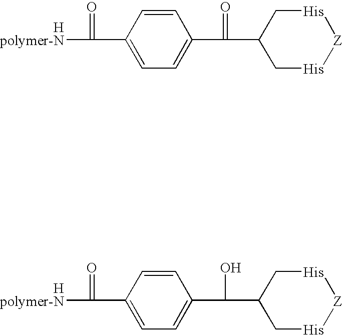

Some examples of reagents which can be used in the process according to the invention include the following:

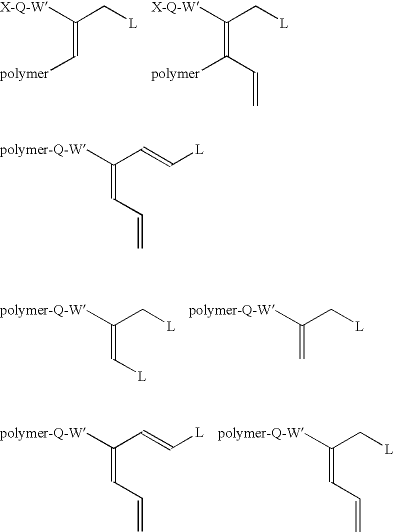

With respect to the protein, the histidine residues are positioned in close proximity to each other, preferably adjacent to each other, either in the native protein or in a polyhistidine tag. Of course, polymers other than polyethylene glycol may replace the PEG in the above formulae. Without wishing to be bound by theory, the bonding to the histidine residues may be as shown in the following formula:

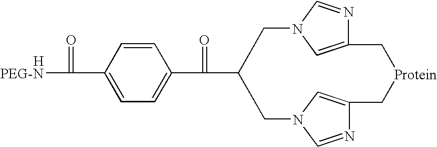

but bonding to other nitrogen atoms in the histidines is also possible.

The immediate product of the above process is a compound of the general formula Ia in which W is an electron-withdrawing group. Such compounds have utility in themselves; because the process of the invention is reversible under suitable conditions, additionally compounds of formula Ia in which W is an electron-withdrawing moiety have utility in applications where release of the free protein is required, for example in direct clinical applications. An electron-withdrawing moiety W may, however, be reduced to give a moiety which prevents release of the protein, and such compounds will also have utility in many clinical, industrial and diagnostic applications. Further, the fact that once W has been reduced reverse reactions can no longer occur, means that no exchange will be observed if a stronger nucleophile is added. Therefore, it becomes possible, for example, to PEGylate via a polyhistidine tag; reduce W; then reduce a disulfide bond in the protein; and carry out a subsequent PEGylation across the reduced disulfide bond. For some proteins, it may be possible to carry out such a process without reducing the group W.

Thus, for example, a moiety W containing a keto group may be reduced to a moiety W containing a CH(OH) group; an ether group CH.OR may be obtained by the reaction of a hydroxy group with an etherifying agent; an ester group CH.O.C(O)R may be obtained by the reaction of a hydroxy group with an acylating agent; an amine group CH.NH₂, CH.NHR or CH.NR₂ may be prepared from a ketone or aldehyde by reductive amination); or an amide CH.NHC(O)R or CH.N(C(O)R)₂ may be formed by acylation of an amine). A group X-Q-W— which is a cyano group may be reduced to an amine group.

The process may be carried out in a solvent or solvent mixture in which all reactants are soluble. The protein may be allowed to react directly with the compound of the general formula II or III in an aqueous reaction medium. This reaction medium may also be buffered, depending on the pH requirements of the nucleophile. The optimum pH for the reaction will generally be at least 6.0, typically between about 6.8 and about 8.5, for example about 6.5 or 7.0 to 8.0, for example about 7.5-8.0 but preferably about 7.0 to 7.5. It is surprising that the process according to the invention is selective to binding to histidine residues (particularly in a polyhistidine tag) at basic pH, as literature methods of PEGylation tend to suggest that basic conditions lead to non-selective PEGylation, including PEGylation to lysine residues, and that PEGylation to histidine is optimised at pH 6.0 to 6.5. Even then, literature methods suggest non-selective PEGylation, including PEGylation to lysine. The optimal reaction conditions will of course depend upon the specific reactants employed, but in general, use of a pH towards the lower end of the above range will tend to increase selectivity towards histidine binding, while use of a pH towards the upper end of the above range will tend to increase the yield.

Reaction temperatures between 3-37° C. are generally suitable: proteins may decompose or denature impairing function if the conjugation reaction is conducted at a temperature where these processes may occur. Reactions conducted in organic media (for example THF, ethyl acetate, acetone) are typically conducted at temperatures up to ambient.

The protein can be effectively conjugated with the desired reagent using a stoichiometric equivalent or a slight excess of reagent, unlike many other reagents. However, since the reagents do not undergo competitive reactions with aqueous media used to solvate proteins, it is possible to conduct the conjugation reaction with an excess stoichiometry of reagent. The excess reagent and the product can be easily separated by ion exchange chromatography during routine purification of proteins, or by separation using nickel.

Another class of compounds useful for conjugating to a polyhistidine tag is represented by a compound of the general formula:

X-[Q-W′—(CH═CH)ᵥ—(CH₂)₂-L]_{v′} (IV)

in which X, Q, v and v′ have the meanings given above, W′ represents an electron withdrawing group, and L represents a leaving group. The direct product resulting from such a conjugation process is a compound of formula Ib in which W is an electron withdrawing group. If desired, this resulting compound of formula Ib can be converted into any other desired product. Specifically, an electron withdrawing group may be reduced, as discussed above.

A particularly preferred reagent of the general formula IV has the general formula IVa:

X—[NH—CO—Ar—W′—(CH═CH)ᵥ—(CH₂)₂—SO₂R]_{v′} (IVa)

in which Ar represents an unsubstituted or substituted aryl group, especially a phenyl group, in which the optional substituents are selected from those mentioned above for an aryl group contained in linking group Q; and R, W′, v and v′ have the meanings given above; to produce a novel conjugate of the general formula:

X—[NH—CO—Ar—W—(CH═CH)ᵥ—(CH₂)₂-Z]_{v′} (Ic)

In these preferred compounds, preferably v is 0, and preferably v′ represents an integer of from 1 to 4, especially 1. Preferably W′ represents a CO group, and W represents a CO group or a CH.OH group. Preferably R represents a C₁₋₄alkyl-aryl group, especially p-tolyl. Preferably Ar is an unsubstituted phenyl group. Preferably X is a polyalkylene glycol, especially polyethylene glycol.

Suitable reaction conditions for use when working with reagents of the general formula IV are the same as those discussed above in the context of reagents II and III.

It is believed that the conjugation process using a reagent of formula IV proceeds via the formation of an intermediate compound of the general formula

X-[Q-W′—(CH═CH)ᵥ—CH═CH₂]_{v′} (V)

in which X, Q, W′, v and v′ have the meanings given above. If it is preferred to generate the compound of the formula V above in situ in the presence of the molecule to be conjugated, a relatively high pH is suitably used throughout. Alternatively, if it is preferred to generate the compound of the formula V above in a separate step and subsequently add the molecule to be conjugate, the first step is suitably carried out at a relatively high pH (e.g. 7.5 to 8.0) while the subsequent step is suitably carried out at a lower pH (e.g. 6.0 to 6.5).

As mentioned above, depending on the number of histidine residues present, more than one polymer may be conjugated to a suitable protein, and this is shown schematically in FIG. 3. For example, two polymers could be conjugated to a 6-residue polyhistidine tag. This provides a useful method of attaching multiple polymer residues to a protein. This may be desired when it is wished to obtain a high molecular weight product using a lower molecular weight PEG, e.g. to obtain a 60 kD molecular weight product using three 20 kD PEG chains; or when it is desired to add two different polymers, for example a protein and a PEG, to another protein to obtain multiple effects; or to achieve, for example, glycosylation and PEGylation in a single step. Such multiple conjugation is often difficult to achieve with conventional conjugation technology.

Many proteins contain free cysteine residues and/or disulfide bridges, and the conjugation reagents used to prepare the novel conjugates of the present invention may also react with exposed cysteine residues and reduced disulfide bridges. Depending upon the reaction conditions and the structure of the protein, they may react with such moieties in preference to reacting with histidine residues in a polyhistidine tag. Therefore, if it is desired to conjugate a protein which contains such moieties as well as a polyhistidine tag, suitable blocking means may be used if required. For example, such moieties may be blocked by conjugation with a reagent corresponding to the general formula II, III or IV given above in which the polymer is a relatively low molecular weight moiety. Conjugation to the histidine residues may then be carried out using the desired reagent.

The compounds of the general formula I have a number of applications. They may for example be used for direct clinical application to a patient, and accordingly, the present invention further provides a pharmaceutical composition comprising a compound of the invention together with a pharmaceutically acceptable carrier. The invention further provides a compound of the invention for use in therapy, and a method of treating a patient which comprises administering a pharmaceutically-effective amount of a compound or a pharmaceutical composition according to the invention to the patient. Any desired pharmaceutical effect, for example trauma treatment, enzyme replacement, protein replacement, wound management, toxin removal, anti-inflammatory, anti-infective, immunomodulatory, vaccination or anti-cancer, may be obtained by suitable choice of protein.

The compounds of the invention may also be used in non-clinical applications. For example, many physiologically active compounds such as enzymes are able to catalyse reactions in organic solvents, and compounds of the invention may be used in such applications. Further, compounds of the invention may be used as diagnostic tools. Compounds of the invention may include an imaging agent, for example a radio nucleotide, to enable tracking of the compound in vivo.

The protein may for example be a peptide, polypeptide, antibody, antibody fragment, enzyme, cytokine, chemokine, receptor, blood factor, peptide hormone, toxin, transcription protein, or multimeric protein.

The following gives some specific proteins which may have utility in the present invention, depending upon the desired application. Enzymes include carbohydrate-specific enzymes, proteolytic enzymes and the like. Enzymes of interest, for both industrial (organic based reactions) and biological applications in general and therapeutic applications in particular include the oxidoreductases, transferases, hydrolases, lyases, isomerases and ligases disclosed by U.S. Pat. No. 4,179,337. Specific enzymes of interest include asparaginase, arginase, adenosine deaminase, superoxide dismutase, catalase, chymotrypsin, lipase, uricase, bilirubin oxidase, glucose oxidase, glucuronidase, galactosidase, glucocerebrosidase, glucuronidase, glutaminase

The proteins used in compounds of the present invention include for example factor VII, VIII or IX and other blood factors, insulin, ACTH, glucagen, somatostatin, somatotropins, thymosin, parathyroid hormone, pigmentary hormones, somatomedins, erythropoietin, luteinizing hormone, hypothalamic releasing factors, antidiuretic hormones, prolactin, interleukins, interferons, colony stimulating factors, hemoglobin, cytokines, antibodies, chorionicgonadotropin, follicle-stimulating hormone, thyroid-stimulating hormone and tissue plasminogen activator.

Certain of the above proteins such as the interleukins, interferons and colony stimulating factors also exist in non-glycosylated form, usually the result of preparation by recombinant protein techniques. The non-glycosylated versions may be used in the present invention.

Other proteins of interest are allergen proteins disclosed by Dreborg et al Crit. Rev. Therap. Drug Carrier Syst. (1990) 6 315 365 as having reduced allergenicity when conjugated with a polymer such as poly(alkylene oxide) and consequently are suitable for use as tolerance inducers. Among the allergens disclosed are Ragweed antigen E, honeybee venom, mite allergen and the like.

Glycopolypeptides such as immunoglobulins, ovalbumin, lipase, glucocerebrosidase, lectins, tissue plasminogen activator and glycosylated interleukins, interferons and colony stimulating factors are of interest, as are immunoglobulins such as IgG, IgE, IgM, IgA, IgD and fragments thereof.

Of particular interest are receptor and ligand binding proteins and antibodies and antibody fragments which are used in clinical medicine for diagnostic and therapeutic purposes. The antibody may used alone or may be covalently conjugated (“loaded”) with another atom or molecule such as a radioisotope or a cytotoxic/anti-infective drug. Epitopes may be used for vaccination to produce an immunogenic polymer-protein conjugate.

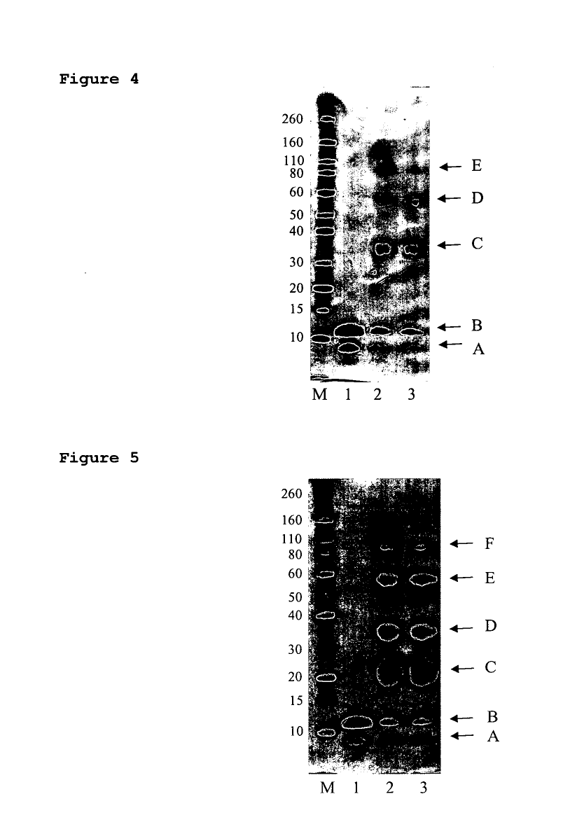

The following Examples illustrate the invention. Results of the Examples are given in the accompanying FIGS. 4 to 20. In the Examples, proteins pro-BNP containing a polyhistidine tag of 6 histidine residues, and beta synuclein containing a polyhistidine tag of 8 histidine residues were used. In cardiac tissue, brain natriuretic peptide (BNP) is synthesized as a 134 amino acid precursor (prepro-BNP), which is cleaved by proteases to form a 108 amino acid pro-BNP. Beta synuclein is a small, soluble protein found primarily in brain tissue, and has a chaperone-like activity. Other proteins used are Interferon α-2b, endostatin, an anti-TNF alpha domain antibody fragment.

### EXAMPLE 1

### 12 kDa PEGylation of His₆-proBNP

### PEG Reagent Structures

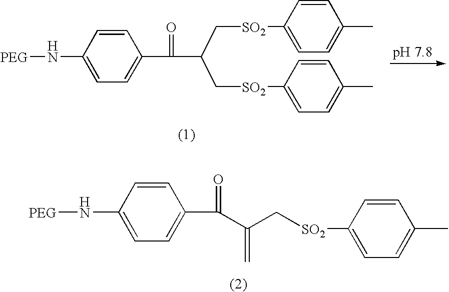

12 kDa PEG mono-sulfone (structure (2)) was prepared by incubation of the PEG bis-sulfone (structure (1)) in pH 7.8, 50 mM sodium phosphate buffer (10 mg/mL) for 5 hours. Two aliquots of C-terminal 6× histidine tagged ProBNP (Abcam, ab51402, 13 kDa) in pH 7.8, 50 mM sodium phosphate (10 μL, 0.5 mg/mL) were chilled on ice and then either 1 molar equivalent (with respect to protein concentration) or 3 molar equivalents of 12 kDa PEG mono-sulfone were added (1.6 μL or 4.8 μL respectively). The reaction mixtures were then placed in the fridge and left for 16 hours.

The resulting reaction mixtures were analysed using SDS-PAGE for size-dependent separation of their constituents and the resulting gel after staining with Instant blue stain (Novexin) is shown in FIG. 4. In FIG. 4, the lane labelled M shows the Novex Sharp protein markers (Invitrogen) used as calibrants. The band labelled B in lane 1 is the sample of His₆-proBNP as supplied (Abcam, 1 mg/mL). Band A is an impurity. Lane 2 is obtained from the 1 equivalent of 12 kDa PEG reaction mixture and shows bands, labelled C, D and E which correspond to a mono-PEGylated product, di-PEGylated product and tri-PEGylated product respectively. The lower bands, labelled A and B correspond to unreacted His₆-proBNP and the impurity in the supplied sample respectively. Lane 3 is obtained from the 3 equivalents of 12 kDa PEG reaction mixture and shows bands, labelled C, D and E which correspond to a mono-PEGylated product, di-PEGylated product and tri-PEGylated product respectively. The lower bands, labelled B and A correspond to unreacted His₆-proBNP and the impurity in the supplied sample respectively.

The gel was then also stained with barium iodide for visualisation of PEG with the result shown in FIG. 5. The bands in Lane 1 correspond to unreacted PEG reagent which is labelled C, and bands labelled D, E and F which correspond to a mono-PEGylated product, di-PEGylated product and tri-PEGylated product respectively. The lower bands, labelled B and A correspond to unreacted His₆-proBNP and the impurity in the supplied sample respectively. Lane 3 which is obtained from the 3 equivalents of 12 kDa PEG reaction mixture shows a band labelled C which corresponds to unreacted PEG reagent and bands labelled D, E and F which correspond to a mono-PEGylated product, di-PEGylated product and tri-PEGylated product respectively. The lower bands, labelled B and A correspond to unreacted His₆-proBNP and the impurity in the supplied sample respectively.

### EXAMPLE 2

### 30 kDa PEGylation of His₆-proBNP

30 kDa PEG mono-sulfone (structure (2) from example 1) was prepared by incubation of the PEG bis-sulfone (structure (1) from example 1) in pH 7.8, 50 mM sodium phosphate buffer (10 mg/mL) for 5 hours. Two aliquots of C-terminal 6× histidine tagged ProBNP (Abcam, ab51402) in pH 7.8, 50 mM sodium phosphate (10 μL, 0.5 mg/mL) were chilled on ice then either 1 molar equivalent (with respect to protein concentration) or 3 equivalents of 30 kDa PEG mono-sulfone were added (2.0 μL or 6.0 μL respectively). The reaction was then placed in the fridge and left for 16 hours.

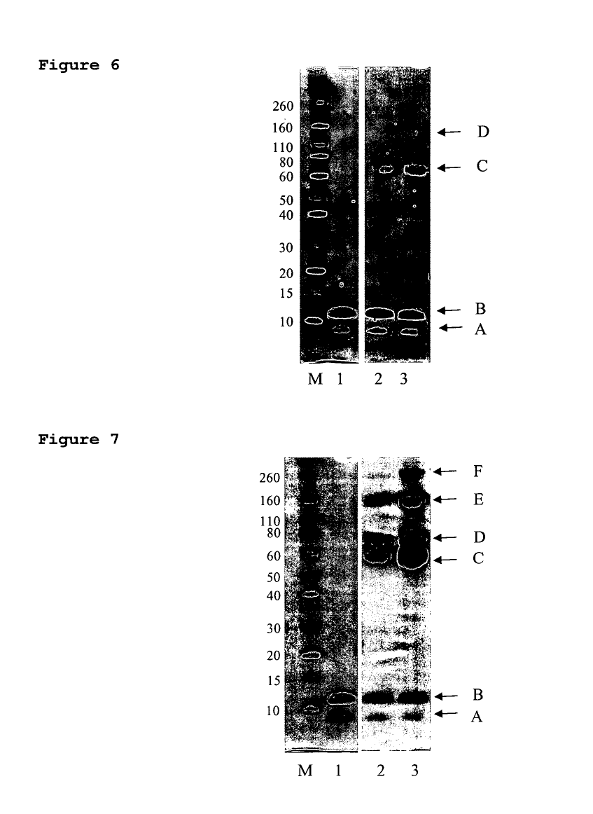

The resulting reaction mixtures were analysed using SDS-PAGE for size-dependent separation of its constituents and the resulting gel after staining with Instant blue (Novexin) is shown in FIG. 6. The lane labelled M shows the Novex Sharp protein markers (Invitrogen) used as calibrants. The band labelled B in lane 1 is the sample of His₆-proBNP as supplied (Abcam, 1 mg/mL). Band A is an impurity. The lane labelled 2 is obtained from the 1 equivalent of 30 kDa PEG reaction mixture and shows a bands labelled C which corresponds to a mono-PEGylated product and lower bands, labelled A and B which correspond to unreacted His₆-proBNP and the impurity in the supplied sample respectively. The lane labelled 3 is obtained from the 3 equivalent of 30 kDa PEG reaction mixture and shows bands, labelled C, and D which correspond to a mono-PEGylated product and di-PEGylated product respectively and lower bands, labelled B and A which correspond to unreacted His₆-proBNP and the impurity in the supplied sample respectively.

The gel was then also stained with barium iodide for visualisation of PEG and the resulting gel is shown in FIG. 7. The lane labelled M shows the Novex Sharp protein markers (Invitrogen) used as calibrants. The band labelled B in lane 1 is the sample of His₆-proBNP as supplied (Abcam, 1 mg/mL). Band A is an impurity. The lane labelled 2 is obtained from the 1 equivalent of 30 kDa PEG reaction mixture and shows a band labelled C which corresponds to unreacted PEG reagent, a band labelled F which corresponds to a PEG impurity, bands labelled D and E and F which correspond to mono-PEGylated and di-PEGylated products respectively, and lower bands, labelled B and A which correspond to unreacted His₆-proBNP and the impurity in the supplied sample respectively. The lane labelled 3 is obtained from the 3 equivalents of 30 kDa PEG reaction mixture and shows a band labelled C which corresponds to unreacted PEG reagent, a band labelled F which corresponds to a PEG impurity, bands labelled D and E which correspond to mono-PEGylated and di-PEGylated products respectively, and lower bands, labelled B and A which correspond to unreacted His₆-proBNP and the impurity in the supplied sample respectively.

### EXAMPLE 3

### 12 kDa PEGylation of His₈-Beta Synuclein and Comparative Reaction with Non-Histidine Tagged Beta Synuclein

12 kDa PEG mono-sulfone (structure (2) from example 1) was prepared by incubation of the PEG bis-sulfone (structure (1) from example 1) in pH 7.8, 50 mM sodium phosphate buffer (10 mg/mL) for 5 hours. Two aliquots of N-terminal 8× histidine tagged beta synuclein (Abcam cat. no. ab40545, kDa) in pH 7.8, 50 mM sodium phosphate (10 μL, 0.39 mg/mL) were chilled on ice then either 1 molar equivalent (with respect to protein concentration) or 3 equivalents of 12 kDa PEG mono-sulfone were added (1.6 μL or 4.7 μL respectively). The reaction was then placed in the fridge and left for 16 hours.

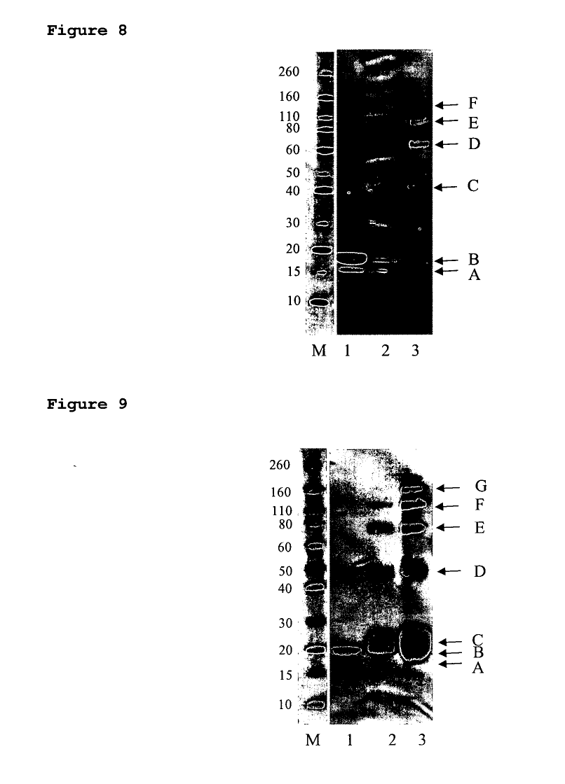

The resulting reaction mixtures were analysed using SDS-PAGE for size-dependent separation of its constituents and the resulting gel after staining with Instant blue (Novexin) is shown in FIG. 8. The lane labelled M shows the Novex Sharp protein markers (Invitrogen) used as calibrants.

The band labelled B in lane 1 is the sample of His₈-beta synuclein supplied by Abcam (0.8 mg/mL). Band A is an impurity. The lane labelled 2 is obtained from the 1 equivalent of 12 kDa PEG reaction mixture and shows bands, labelled C and D which correspond to a mono-PEGylated product and di-PEGylated product respectively and lower bands, labelled A and B which correspond to unreacted His₈-beta synuclein and the impurity in the supplied sample respectively. The lane labelled 3 is obtained from the 3 equivalents of 12 kDa PEG reaction mixture and shows bands, labelled C, D, E and F which correspond to a mono-PEGylated product, di-PEGylated product, tri-PEGylated product and tetra-PEGylated product respectively and lower bands, labelled B and A which correspond to unreacted His₈-beta synuclein and the impurity in the supplied sample respectively.

The gel was then also stained with barium iodide for visualisation of PEG and the resulting gel is shown in FIG. 9. In FIG. 9, the lane labelled M shows the Novex Sharp protein markers (Invitrogen) used as calibrant. The lane labelled 1 is obtained from His₈-beta synuclein as supplied by Abcam. The lane labelled 2 is obtained from the 1 equivalent of 12 kDa PEG reaction mixture and shows bands labelled C, D and E which correspond to a mono-PEGylated product, di-PEGylated product and tri-PEGylated product respectively, and lower bands, labelled B and A which correspond to unreacted His₈-beta synuclein and the impurity in the supplied sample respectively. The lane labelled 3 is obtained from the 3 equivalents of 12 kDa PEG reaction mixture and shows bands labelled D, E and F which correspond to a mono-PEGylated product, di-PEGylated product, tri-PEGylated product and tetra-PEGylated product respectively, and lower bands, labelled B and A which correspond to unreacted His₈-beta synuclein and the impurity in the supplied sample respectively. Band C is a PEG impurity.

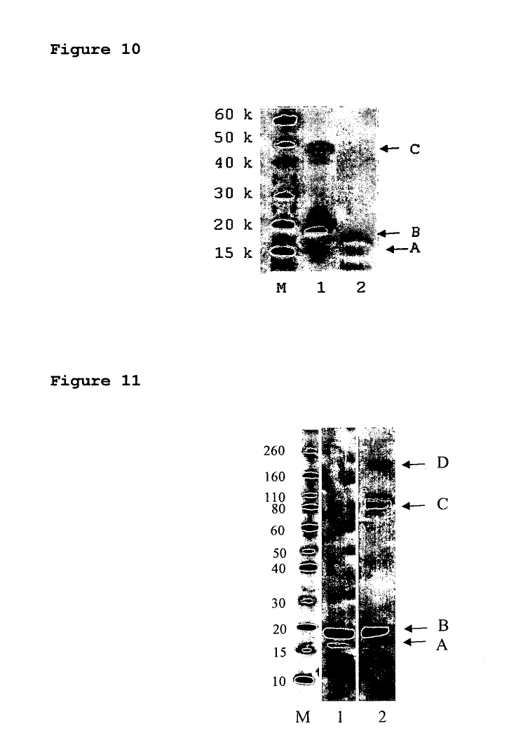

A comparison 12 kDa PEGylation study was performed between His₈-beta synuclein and non-histidine tagged beta synuclein (Abcam. cat no. ab48853) using 1 equivalent of the PEG reagent (2) at pH 7.0 and the SDS-PAGE result is shown in FIG. 10. The lane labelled M shows the Novex Sharp protein markers (Invitrogen) used as calibrants. The His₈-beta synuclein result is shown in the lane labelled 1. In lane 1, the band labelled B is unreacted His₈-beta synuclein and the band labelled C is mono-PEGylated His₈-beta synuclein. The lane labelled 2 shows the non-histidine tagged beta synuclein reaction and the only visible band is labelled A and is the non-histidine tagged beta synuclein. There is no PEGylated protein band in lane 2.

### EXAMPLE 4

### 30 kDa PEGylation of His₈-Beta Synuclein

30 kDa PEG mono-sulfone (structure (2) from example 1) was prepared by incubation of the PEG bis-sulfone (structure (1) from example 1) in pH 7.8, 50 mM sodium phosphate buffer (10 mg/mL) for 5 hours. An aliquot of N-terminal 8× histidine tagged beta synuclein (Abcam, ab40545, 15 kDa) in pH 7.8, 50 mM sodium phosphate (10 μL, 0.39 mg/mL) was chilled on ice then 1 molar equivalent (with respect to protein concentration) of 30 kDa PEG mono-sulfone was added (1.6 μL). The reaction was then placed in the fridge and left for 16 hours.

The resulting reaction mixture was analysed using SDS-PAGE for size-dependent separation of its constituents and the resulting gel after staining with Instant blue (Novexin) is shown in FIG. 11. In FIG. 11, the lane labelled M shows the Novex Sharp protein markers (Invitrogen) used as calibrants. The lane labelled 1 is obtained from the sample of His₈-beta synuclein supplied by Abcam (0.8 mg/mL) and the band labelled B is the Polyhistidine tagged protein whilst the band labelled A is an impurity. The lane labelled 2 is obtained from the 1 equivalent of 30 kDa PEG reaction mixture and shows a band labelled C which corresponds to a mono-PEGylated product and lower bands, labelled A and B which correspond to unreacted His₈-beta synuclein and the impurity in the supplied sample respectively.

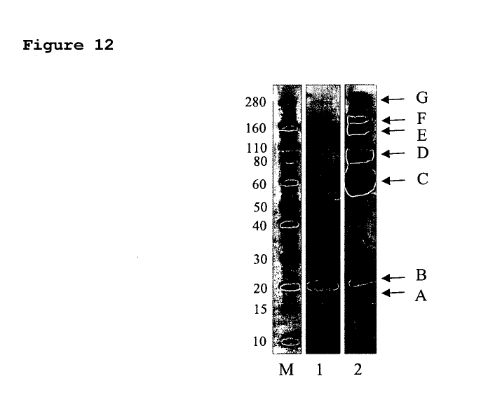

The gel was then also stained with barium iodide for visualisation of PEG and the resulting gel is shown in FIG. 12. In FIG. 12, the lane labelled M shows the Novex Sharp protein markers (Invitrogen) used as calibrants. The lane labelled 1 is obtained from His₈-beta synuclein as supplied by Abcam. The lane labelled 2 is obtained from the 1 equivalent of 30 kDa PEG reaction mixture and shows a band labelled C which corresponds to unreacted PEG reagent, bands labelled E and G which corresponds to PEG impurities, bands labelled D and F which correspond to a mono-PEGylated product and di-PEGylated product respectively, and lower bands, labelled B and A which correspond to unreacted His₈-beta synuclein and the impurity in the supplied sample respectively.

### EXAMPLE 5

### PEGylation on Histidine in IFN α-2b with an 8 Histidine Sequence on its C Terminal with 10 kDa and 20 kDa PEG Reagent 3

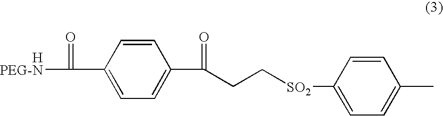

### Step 1, Synthesis of p-carboxy-3-piperidinopropriophenone hydrochloride 3(2)

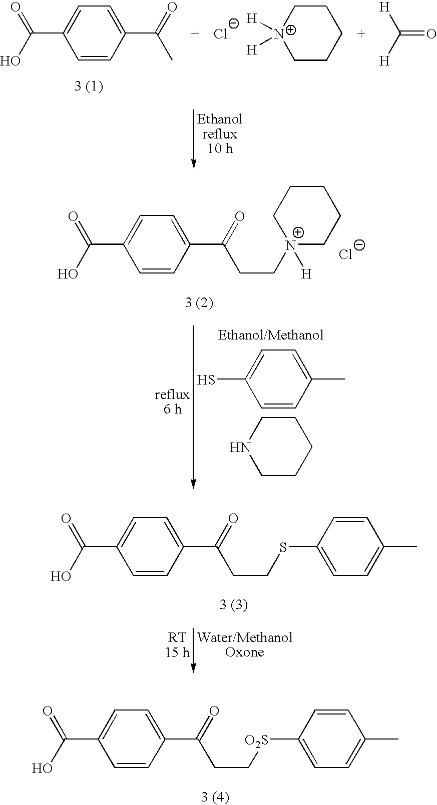

A 250 ml round bottom flask was charged with p-acetylbenzoic acid (15.0 g, 3(1)), 11.11 g piperidine hydrochloride and 8.23 g paraformaldehyde. Absolute ethanol (90 ml) and concentrated hydrochloric acid (1 ml) were then added, and the resulting suspension heated under reflux for 10 h while stirring under argon. After stopping the reflux, acetone (150 ml) was added and the reaction mixture allowed to cool to room temperature. The resulting white precipitate was isolated on a glass filter (G3) and washed twice with chilled acetone. The solid was then dried under vacuum to give a white crystal powder (3(2), 9.72 g). ¹H NMR (400 MHz, DMSO-d₆) δ 1.79, 2.96, 3.45 (br m, CH₂ of piperidine moiety), 3.36 (t, 2H, COCH₂), 3.74 (t, 2H, NCH₂), 8.09 (m, 4H, ArH).

### Step 2: Synthesis of 4-(3-(p-tolylthio)propanoyl)benzoic acid 3(5)

The p-carboxy-3-piperidinopropriophenone hydrochloride 3(2) (1.0 g) and 4-methylbenzenethiol (417 mg, 3(3)) were suspended in a mixture of absolute ethanol (7.5 ml) and methanol (5 ml). Piperidine (50 μl) was then added and the suspension heated to reflux with stirring for 6 h in an argon atmosphere. The white precipitate afforded after cooling to room temperature was filtered off with a glass filter (G3), washed carefully with cold acetone and dried in vacuum to give 3(3) (614 mg). ¹H NMR (400 MHz, DMSO-d₆) δ 2.27 (s, 3H, phenyl-CH₃), 3.24, 3.39 (t, 2×2H, CH₂), 7.14, 7.26 (d, 2×2H, ArH of tolyl moiety), 8.03 (m, 4H, ArH of carboxylic acid moiety).

### Step 3, Synthesis of 4-(3-tosylpropanoyl)benzoic acid 3(4)

4-(3-(p-tolylthio)propanoyl)benzoic acid 3(4) (160 mg) was suspended in a mixture of water (10 ml) and methanol (10 ml). After cooling in an ice bath, oxone (720 mg, Aldrich) was added and the reaction mixture allowed to warm to room temperature while stirring overnight (15 h). The resulting suspension was diluted with further water (40 ml) so that it became nearly homogeneous and the mixture was then extracted three times with chloroform (in total 100 ml). The pooled chloroform extracts were washed with brine and then dried with MgSO₄. Evaporation of volatiles under vacuum at 30° C. afforded a white solid 3(4) (149 mg). ¹H NMR (400 MHz, DMSO-d₆) δ 2.41 (s, 3H, phenyl-CH₃), 3.42 (t, 2H, CO—CH₂), 3.64 (t, 2H, SO₂—CH₂), 7.46, 7.82 (d, 2×2H, ArH of tolyl moiety), 8.03 (m, 4H, ArH of carboxylic acid moiety).

### Step 4, Synthesis of PEGylated 4-(3-tosylpropanoyl)benzoic acid, PEG Reagent 3

The 4-(3-tosylpropanoyl)benzoic acid 3(4) (133 mg) and O-(2-aminoethyl)-O′-methyl-PEG (MW 10 kDa, 502 mg, BioVectra) were dissolved in dry toluene (5 ml). The solvent was removed under vacuum without heating and the dry solid residue was then redissolved in dry dichloromethane (15 ml) under argon. To the resulting solution, cooled in an ice bath, was slowly added diisopropylcarbodiimide (DIPC, 60 mg) under argon. The reaction mixture was then kept stirring at room temperature overnight (15 h). Volatiles were then removed under vacuum (30° C., water bath) to afford a solid residue that was redissolved with gentle heating (35° C.) in acetone (20 ml). The solution was filtered over non-absorbent cotton wool to remove insoluble material. The solution was then cooled in a dry ice bath to give a white precipitate that was separated by centrifugation (4600 rpm, 30 min). The liquid phase was decanted off and this precipitation procedure was repeated three times. Afterwards the resulting off-white solid was dried under vacuum to afford the PEG reagent 3 (437 mg). ¹H NMR (400 MHz, CDCl₃) δ 2.46 (s, 3H, phenyl-CH₃), 3.38 (s, 3H, PEG-OCH₃), 3.44-3.82 (br m, PEG), 7.38, 7.83 (d, 2×2H, ArH of tolyl moiety), 7.95 (m, 4H, ArH of carboxylic acid moiety).

### Step 5: PEGylation on Histidine of C-Terminal His₈-Tagged IFN α-2b

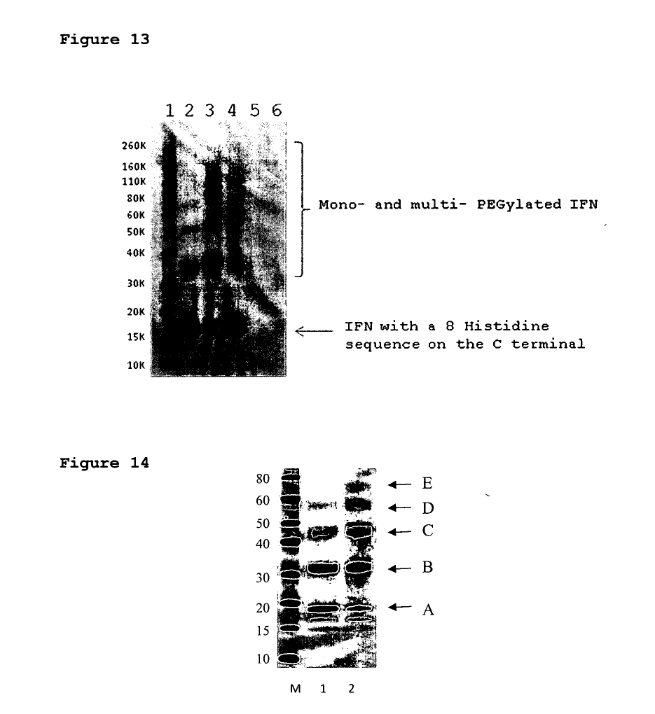

To a 20 μl solution of IFN α-2b (1.13 mg/ml in 10 mM sodium phosphate buffer containing 2 mM EDTA and 150 mM NaCl, pH 7.5) was added 1 molar equivalent of 10 kDa PEG reagent 3 (1.8 μl of a 6 mg/ml solution in deionised water) and the resulting solution incubated overnight at room temperature. A repeat was also performed with 1 molar equivalent of 20 kDa PEG reagent, again prepared by a method analogous to that as given above (3.3 μl of a 6.6 mg/ml solution in deionised water. Both samples were then analysed by SDS-PAGE (NuPAGE® Novex 4-12% Bis-Tris gels, MES running buffer, all from Invitrogen, and InstantBlue stain (Expedeon cat. No. ISB1L)). The result is shown in FIG. 13. In the lane labelled 1 are the protein markers. Lane 2 is the starting IFN only. Lane 3 shows the result of the 10 kDa PEG reagent reaction. There are 5 distinct bands between the 30 and 160 KDa protein markers corresponding to IFN with 1 to 5 PEG chains conjugated. Lane 4 shows the result of the 20 kDa PEG reagent reaction. There are three distinct bands between the 60 to 110 KDa protein markers corresponding to IFN with 1 to 3 PEG chains conjugated. The lane labelled 5 is the 20 kDa PEG reagent which does not stain, so no band is visible. The lane labelled 6 is the 10 kDa PEG reagent which does not stain, so no band is visible.

### EXAMPLE 6

### 5 kDa PEGylation of His₈-β-Synuclein

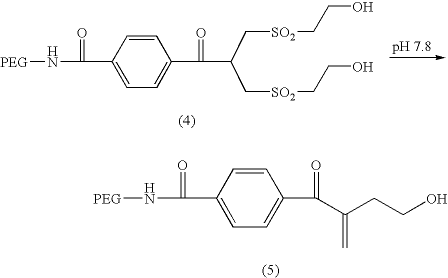

5 kDa PEG mono-sulfone (structure (5)) was prepared by incubation of the PEG bis-sulfone (structure (4)) in pH 7.8, 50 mM sodium phosphate buffer (5 mg/mL) for 3 hours. Two aliquots of N-terminal 8× histidine tagged beta synuclein (Abcam cat. no. ab40545, 15.4 kDa) in pH 7.4, 50 mM sodium phosphate (10 μL, 0.38 mg/mL) were chilled on ice then either 1 molar equivalent (with respect to protein concentration) or 3 equivalents of 5 kDa PEG mono-sulfone (5) were added (0.25 μL or 0.74 μL respectively). As control, two aliquots of beta synuclein (Abcam cat. no. ab48853, 14.3 kDa) were treated the same way and given either 1 molar equivalent (0.18 μL) or 3 equivalents (0.52 μL) of the 5 kDa PEG mono-sulfone. The reactions were then incubated at ambient temperature for 6 h. The resulting reaction solutions were analysed using SDS-PAGE and stained with Instant blue (Expedeon) as shown in FIG. 14. The lane labelled M shows the Novex Sharp protein markers (Invitrogen) used as calibrants. The lanes labelled 1 and 2 are obtained from the 1 equivalent of 5 kDa PEG reaction mixture and 3 equivalents of PEG reaction mixture respectively. The band labelled A is His₈-beta synuclein.

Faint bands below it are impurities from the protein supplied by Abcam. The bands, labelled B, C, D and E which correspond to mono-PEGylated, di-PEGylated, tri-PEGylated and tetra-PEGylated protein products respectively.

### EXAMPLE 7

### 10 kDa PEGylation of His₆-Anti TNF-α Domain Antibody Fragment at Different pH's

10 kDa PEG mono-sulfone (structure (2) from example 1) was prepared by incubation of the PEG bis-sulfone (structure (1) from example 1, 10 mg/mL) in pH 7.8, 50 mM sodium phosphate buffer (5 mg/mL) for 3 h. The C-terminal 6× histidine tagged anti-TNF alpha domain antibody fragment (12.7 kDa) solution (0.6 mg/mL) in 50 mM sodium phosphate, 150 mM sodium chloride and 2 mM EDTA was prepared at 4 different pH's (pH 6.2, pH 6.7, pH 7.0 and pH 7.4). Three molar equivalents of 10 kDa PEG mono-sulfone ((2), 1.41 μL) were added to the His₆-domain solutions at each of the four different pH's (10 μL, 0.6 mg/mL). The reactions were then incubated at ambient temperature for 3 hours.

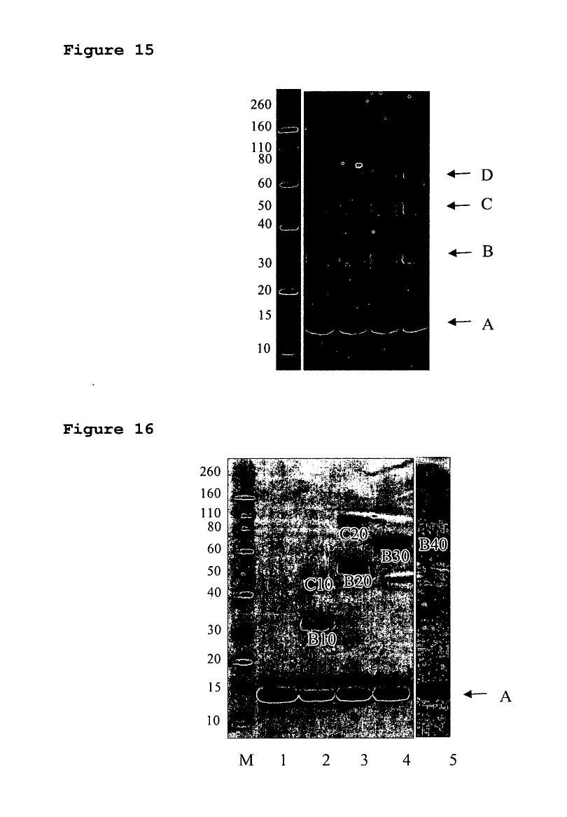

The resulting reaction solutions were analysed using SDS-PAGE and stained with Instant blue (Expedeon) as shown in FIG. 15. The lane labelled M shows the Novex Sharp protein markers (Invitrogen). The lanes labelled 1 to 4 are obtained from the reaction mixture using 3 molar equivalents of 10 kDa PEG at pH 6.2, pH 6.7, pH 7.0 and pH 7.4 respectively. The band labelled A is unreacted His₆-anti-TNF alpha domain antibody fragment. The band labelled B is the mono-PEGylated domain product. The bands labelled C and D correspond to di-PEGylated and tri-PEGylated domain fragment respectively. PEGylation is seen at each of the four pH's and the extent of PEGylation increases as the pH is increased.

### EXAMPLE 8

### PEGylation of His₆-Anti TNF-αDomain Antibody Fragment Using 10 kDa, 20 kDa, 30 kDa and 40 kDa PEG

10 kDa, 20 kDa, 30 kDa and 40 kDa PEG bis-sulfone (structure (1) from example 1) were separately incubated in pH 7.8, 50 mM sodium phosphate buffer for 3 hours at ambient temperature to give the corresponding PEG mono-sulfones (structure (2) from example 1). The concentration of PEG was 10 mg/mL, 20 mg/mL, 30 mg/mL and 40 mg/mL for 10 kDa, 20 kDa, 30 kDa and 40 kDa PEG respectively. Four aliquots of the C-terminal 6× histidine tagged anti-TNF domain antibody (12.7 kDa) solution (1.25 mg/mL, 5 μL) in 50 mM sodium phosphate, 150 mM sodium chloride and 2 mM EDTA were then added with the 10 kDa, 20 kDa, 30 kDa and 40 kDa PEG mono-sulfone solutions (0.74 μL, 1.5 molar equivalents). The reactions were then incubated at 4° C. for 8 hours. The reaction solutions were then analysed by SDS-PAGE as shown in FIG. 16. The Lane labelled M shows the Novex sharp protein markers (Invitrogen) used as calibrants. The lane labelled 1 is the His₆-anti TNF domain antibody fragment shown as a reference. The lanes labelled 2 to 5 are obtained from the reaction of 10 kDa, 20 kDa, 30 kDa and 40 kDa PEGs respectively. The bands labelled A are unreacted His₆-anti-TNF domain. The bands labelled B (B10, B20, B30 and B40) correspond to the mono-PEGylated domain fragments for 10, 20, 30 and 40 kDa PEG respectively. The band labelled C10 corresponds to the di-PEGylated product for the 10 kDa PEG reaction and the C20 band corresponds to the di-PEGylated product for the 20 kDa PEG reaction.

### EXAMPLE 9

### 2 kDa PEGylation of His₆-Endostatin and Comparative Reaction with Endostatin not Possessing a Polyhistidine Tag

2 kDa PEG mono-sulfone (structure (2) from example 1) was prepared by incubation of the PEG bis-sulfone (structure (1)) from example 1) in pH 7.8, 50 mM sodium phosphate buffer (1 mg/mL) for 4 hours. Two aliquots of C-terminal 6× histidine tagged Endostatin (Calbiochem cat. no. 324743) in pH 6.2, 50 mM sodium phosphate (30 μL, 0.5 mg/mL) were chilled on ice then either 1 molar equivalent (with respect to protein concentration) or 3 equivalents of 2 kDa PEG mono-sulfone were added (1.4 μL or 4.2 μL respectively). The reaction mixture was then placed in the fridge and left for 16 hours.

Two aliquots of non-tagged Endostatin (Calbiochem cat. no. 324769) in pH 6.2, 50 mM sodium phosphate (10 μL, 0.2 mg/mL) were also chilled on ice, then either 1 molar equivalent (with respect to protein concentration) or 3 equivalents of 2 kDa PEG mono-sulfone (0.1 mg/mL) were added (2.0 μL or 6.0 μL respectively). The reaction was then placed in the fridge and left for 16 hours.

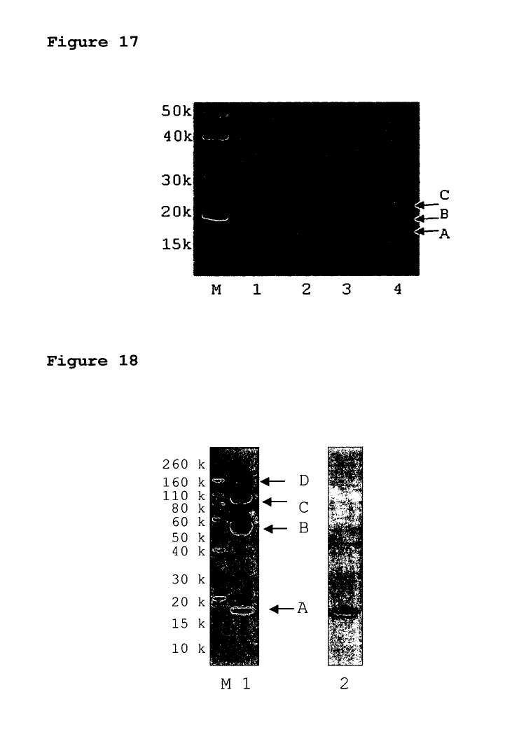

The resulting reaction solutions were analysed using SDS-PAGE for size-dependent separation of their constituents and the resulting gel after staining with Instant blue (Expedeon) is shown in FIG. 17. In FIG. 17, the left-hand column of spots labelled M shows the Novex Sharp protein markers (Invitrogen). The lane labelled 1 is obtained from the sample of the 1 equivalent of 2 kDa PEG with His₆-Endostatin reaction and shows bands, labelled B and C which correspond to the unreacted protein, and mono-PEGylated product respectively. The lane labelled 2 is obtained from the sample of the 3 equivalent of 2 kDa PEG with His₆-Endostatin reaction and shows bands, labelled B and C which correspond to the unreacted protein and mono-PEGylated product respectively. The lanes labelled 3 and 4 were obtained from the 1 equivalent of 2 kDa PEG with Endostatin reaction and 3 equivalents of 2 kDa PEG with Endostatin reaction respectively. Only a single band labelled A and B respectively which corresponds to the unreacted protein is present for both reactions showing that the 2 kDa PEG only reacts with the His₆-Endostatin.

### EXAMPLE 10

### 20 kDa and 30 kDa PEGylation of His₈-Interferon Alpha and Comparison Reaction with Non-Histidine Tagged Interferon Alpha

A solution of N-terminal 8× histidine tagged interferon α-2b (2.63 mL, 1.14 mg/mL) was prepared in pH 7.4, 50 mM sodium phosphate buffer containing 150 mM sodium chloride (PBS) and then 1.7 molar equivalent (with respect to the protein concentration) of 20 kDa PEG bis-sulfone (structure (1) from example 1) was added (420 μL of 11.5 mg/mL 20 kDa PEG bis-sulfone solution in deionised water). The reaction was placed in the refrigerator for 18 hours. The resulting reaction mixture was analysed by SDS-PAGE and the result shown in FIG. 18. In FIG. 18, the lane labelled M shows the Novex Sharp protein markers (Invitrogen) used as calibrants. The lane labelled 1 shows the reaction solution where the band labelled A corresponds to unreacted IFN, the band labelled B to monoPEGylated IFN, the band labelled C to diPEGylated IFN and the band labelled D to triPEGylated IFN.

A second reaction with one equivalent of 30 kDa PEG at 0.5 mg/ml and a solution of N-terminal 8× histidine tagged interferon α-2b (3.2 mL, 0.793 mg/mL) at pH 7.5. The result was similar to the 20 kDa reaction with a mono-PEGylated IFN band visible between the 60 and 80 kDa protein markers, a band between 110 and 160 kDa corresponding to di-PEGylated IFN and a third band between the 160 and 260 kDa protein markers corresponding to tri-PEGylated IFN. A comparison with non-histidine tagged IFN alpha-2b showed no reaction with one equivalent of PEG reagent (1) after 18 h and under the same conditions, with the result shown in lane 2 of FIG. 18.

### EXAMPLE 11

### Use of Poly(Vinyl Pyrrolidone) (PVP Bis-Sulfone) for Conjugation to N-Terminal 8× Histidine Tagged Interferon Alpha

Preparation of PVP with a Terminal Amine Group: a Pressure tube was charged with cysteamine (0.042 g), dioxane (8 ml) and a magnetic stir bar. After gentle heating to allow a solution to form, the solution was purged with argon at room temperature for 5 min. While still purging, 1-vinyl-2-pyrrolidone (2.0 g) was then added and after a further 5 min this was followed by 2,2′-azobis(2-methylpropionitrile) (0.089 g). After a further 2 min the pressure tube was sealed with a screw cap under argon and placed in an oil bath at 60° C. for 23 h with stirring. After allowing the tube and contents to cool to room temperature, diethyl ether (15 ml) was added causing precipitation of the polymeric product. The liquid phase was decanted away and the solid residue redissolved in acetone (3 ml). The resulting acetone solution was then added dropwise to rapidly stirring diethyl ether (25 ml) and the precipitate isolated on a no. 2 sintered glass funnel with a slight burst of vacuum. The solid was washed with fresh diethyl ether (10 ml) and then allowed to dry under vacuum at room temperature (mass=1.24 g, white solid).

Conjugation of protein reactive end group to PVP-amine: PVP-amine (500 mg), 4-[2,2-bis[(p-tolylsulfonyl)methyl]acetyl]-benzoic acid (125 mg), and 4-dimethylaminopyridine (6 mg) were mixed with anhydrous dichloromethane (10 ml) under argon and with stirring was then added 1,3-diisopropylcarbodiimide (80 μl). The resulting mixture was allowed to stir for 20 h at room temperature and then filtered though non-absorbent cotton-wool. To the filtrate was then added diethyl ether (20 ml) and the resulting precipitate isolated by centrifugation (2,000 rpm, 2 min, 2° C.). The liquid phase was decanted off and the remaining residue vortexed with ethyl acetate (5 ml) for several minutes. After decanting off the ethyl acetate phase the solid residue was redissolved in dichloromethane (5 ml) followed by the addition of ethyl acetate (15 ml). The solution was placed in dry-ice for 15 min resulting in precipitation of a solid which was then isolated by centrifugation (2,000 rpm, 2 min, 2° C.). The sticky residue obtained was vortexed with fresh ethyl acetate (5 ml) and then dried under vacuum at room temperature (mass=0.118 g).

Fractionation of PVP bis-sulfone: The solid material obtained from the above step was mixed with aqueous 20 mM sodium acetate buffer, 150 mM NaCl, pH 4.0 and then centrifuged at 13,000 rpm until a clear solution was visible. The solution phase removed from the solid residue and then fractionated on a HiLoad 16/60 Superdex™ 200 prep grade size exclusion column (GE Healthcare) running 20 mM sodium acetate buffer, 150 mM, pH 4.0 at 1 ml/min by collecting fractions every 1 min during peak elution. The fractions eluting between 72 to 80 min was used for protein conjugation.

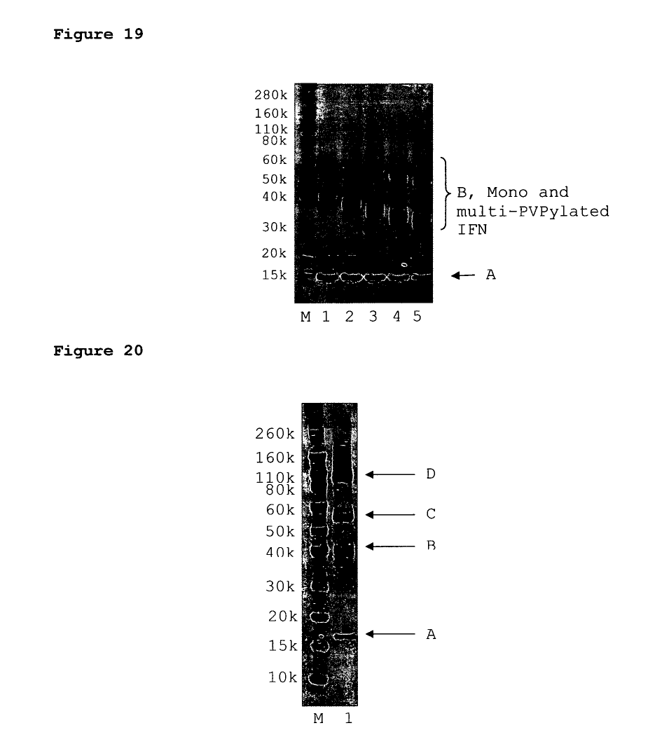

PVP conjugation to N-terminal 8× histidine tagged IFN alpha: IFN (0.025 mg, 0.5 mg/ml) was prepared in 7.5, 50 mM sodium phosphate buffer containing 150 mM sodium chloride (PBS). To 50 μl of IFN (25 μg, 0.5 mg/ml) was added 50 μl of the fractionated PVP bis-sulfone solution (Fractions 72 min, 74 min, 76 min, 78 min and 80 min). The resulting solution was gently mixed and left at 4° C. overnight. The resultant reaction solution was analysed by SDS-PAGE and the result is shown in FIG. 19. In FIG. 19, the lane labelled M shows the Novex Sharp protein markers (Invitrogen) used as calibrants. The lanes labelled 2 to 5 show the reaction solutions from the different sized PVP reagent fractions used (72 min to 80 min PVP fractions from 2 to 5 respectively). The bands labelled B show the mono- and multi-PVPylated IFN obtained.

### EXAMPLE 12

### Multimeric Conjugation of Interferon Alpha with Interferon Alpha Using N-Terminal 8× Histidine Tagged Interferon Alpha

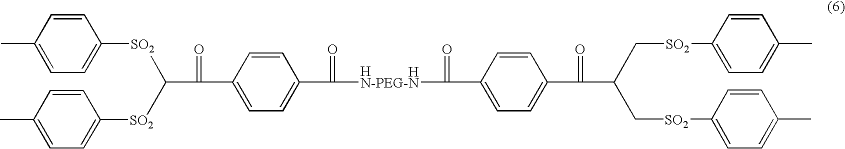

A solution of N-terminal 8× histidine tagged Interferon α-2b (50 μL, 1.14 mg/mL) was prepared in pH 7.5, 50 mM sodium phosphate buffer containing 150 mM sodium chloride (PBS) and then 0.125 molar equivalents (with respect to the protein concentration) of 10 kDa PEG bifunctional PEG reagent (6) was added (0.47 μL of an 8 mg/mL solution of (6) in water). The reaction was left at ambient temperature for 18 hours. The resulting reaction mixture was analysed by SDS-PAGE. The resultant gel showed a band between the 50 kDa and 60 kDa Novex sharp protein markers (Invitrogen) after staining with Instant Blue (Expedeon) that corresponds to the IFN-PEG-IFN fusion conjugate.

### EXAMPLE 13

### Multimeric Conjugation of N-Terminal 8× Histidine Tagged Interferon Alpha with Human Serum Albumin (HSA) Containing a Free Cysteine

To a solution of N-terminal 8× histidine tagged Interferon α-2b (1 mL, 1.14 mg/mL) prepared in pH 7.5, 50 mM sodium phosphate buffer containing 150 mM sodium chloride (PBS) was added human serum albumin (3 molar equivalents to IFN, 10.77 mg) and allowed to dissolve. To this solution was added 10 kDa PEG bis(bis-sulfone) (structure (6) from example 12) (1 molar equivalent to IFN, 75 μL of 8 mg/mL 10 kDa PEG bis (bis-sulfone) (6) solution in water). The reaction mixture was left at ambient temperature for 18 hours. The resulting reaction mixture was purified by ion-exchange chromatography followed by metal-ion affinity chromatography and then analysed by SDS-PAGE and the result is shown in FIG. 20. In FIG. 20, the lane labelled M shows the Novex Sharp protein markers (Invitrogen) used as calibrants. The lane labelled 1 shows the purified reaction solution where the band labelled A is unreacted IFN, the band labelled B is mono-PEGylated IFN, the band labelled C is IFN-PEG-IFN and the band labelled D is IFN-PEG-albumin.

1. A compound comprising a polymer conjugated to a protein or peptide, said conjugation being via a polyhistidine tag.

2. A compound as claimed in claim 1, in which the polyhistidine tag contains from 2 to 12 histidine residues.

3. A compound as claimed in claim 2, in which the polyhistidine tag contains from 4 to 10 histidine residues.

4. A compound as claimed in claim 1, in which the polymer is a polyalkylene glycol, a polyvinylpyrrolidone, a polyacrylate, a polymethacrylate, a polyoxazoline, a polyvinylalcohol, a polyacrylamide, a polymethacrylamide, a HPMA copolymer, a polyester, polyacetal, poly(ortho ester), polycarbonate, poly(imino carbonate), polyamide, a copolymer of an alkylene oxide and a polyester, polyacetal, poly(ortho ester), or poly(amino acid), a polysaccharide, a protein, polyglutamic acid, a copolymer of a saccharide or an amino acid and a synthetic monomer, or a copolymer of divinylether-maleic anhydride and styrene-maleic anhydride.

5. A compound as claimed in claim 4, in which the polymer is a polyalkylene glycol.

6. A compound as claimed in claim 5, in which the polymer is a polyalkylene glycol containing C₂ and/or C₃ units.

7. A compound as claimed in claim 6, in which the polymer is a polyethylene glycol.

8. A compound as claimed in claim 6, in which the polymer is a protein.

9. A compound as claimed in claim 1, in which said conjugated protein or peptide is a peptide, polypeptide, antibody, antibody fragment, enzyme, cytokine, chemokine, receptor, blood factor, peptide hormone, toxin, transcription protein, or multimeric protein.

10. A compound as claimed in claim 9, in which said conjugated protein or peptide is an antibody or an antibody fragment.

11. A compound as claimed in claim 1, in which said conjugated protein or peptide is a native protein which has been reacted with one or more blocking groups to protect sensitive groups thereon; or is a protein which is additionally conjugated with one or more polymers, small molecules which are therapeutics or diagnostics, sialic acid, sugars, and/or starches.

12. A compound as claimed in claim 1, which is a compound of the general formula 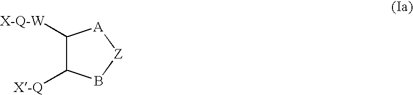
in which one of X and X′ represents a polymer, and the other represents a hydrogen atom;
each Q independently represents a linking group;
W represents an electron-withdrawing moiety or a moiety
preparable by reduction of an electron-withdrawing moiety; or, if X′ represents a polymer, X-Q-W— together may represent an electron withdrawing group; and in addition, if X represents a polymer, X′ and electron withdrawing group W together with the interjacent atoms may form a ring;
Z represents a protein or a peptide linked to A and B via a polyhistidine tag;
A is a C₁₋₅ alkylene or alkenylene chain; and
B is a bond or a C₁₋₄ alkylene or alkenylene chain.

13. A compound as claimed in claim 12, in which each Q independently represents a direct bond, a C₁₋₁₀ alkylene group, or an optionally-substituted aryl or heteroaryl group, any of which may be terminated or interrupted by one or more oxygen atoms, sulphur atoms, —NR groups in which R represents a hydrogen atom or an alkyl or an aryl group, keto groups, —O—CO— groups and/or —CO—O— groups.

14. A compound as claimed in claim 13, in which Q is an alkylene group or a direct bond.

15. A compound as claimed in claim 13, in which W represents a keto or aldehyde group CO, an ester group —O—CO— or a sulphone group —SO₂—, or a group obtained by reduction of such a group; or in which X-Q-W— together represent an electron withdrawing group.

16. A compound as claimed in claim 13, in which X is a polymer, and X′-Q- is H—.

17. A compound as claimed in claim 1, which is a compound of the general formula:
X-[Q-W—(CH═CH)ᵥ—(CH₂)₂-Z]_{v′} (1b)
in which X represents a polymer; Q represents a linking group; W represents an electron-withdrawing group or a moiety preparable by reduction of an electron-withdrawing moiety; v represents 0 or an integer of from 1 to 4; V′ represent an integer of from 1 to 8; and Z represents a protein or a peptide linked via a polyhistidine tag.

18. A compound as claimed in claim 17, in which v is 0.

19. A process for the preparation of a compound as claimed in claim 1, which comprises reacting a polymeric conjugation reagent with a protein or peptide containing a polyhistidine tag under conditions such that conjugation occurs via said polyhistidine tag.

20. A compound of the general formula 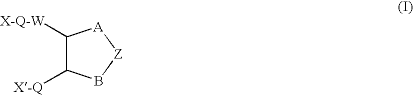
in which one of X and X′ represents a polymer, and the other represents a hydrogen atom;
each Q independently represents a linking group;
W represents an electron-withdrawing moiety or a moiety
preparable by reduction of an electron-withdrawing moiety; or, if X′ represents a polymer, X-Q-W— together may represent an electron withdrawing group; and in addition, if X represents a polymer, X′ and electron withdrawing group
W together with the interjacent atoms may form a ring;
Z represents a protein or a peptide linked to A and B via respective histidine residues;
A is a C₁₋₅ alkylene or alkenylene chain; and
B is a bond or a C₁₋₄ alkylene or alkenylene chain.

21. A compound as claimed in claim 20, in which said respective histidine residues are present in the native protein or peptide.

22. A process for the preparation of a compound as claimed in claim 12, which comprises reacting either (i) a compound of the general formula 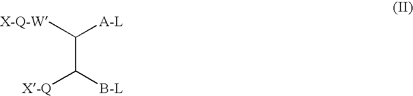
in which one of X and X′ represents a polymer and the other represents a hydrogen atom;
Q represents a linking group;
W′ represents an electron-withdrawing group; or, if X′ represents a polymer, X-Q-W′ together may represent an electron withdrawing group;
A represents a C₁₋₅ alkylene or alkenylene chain;
B represents a bond or a C₁₋₄ alkylene or alkenylene chain; and
each L independently represents a leaving group;
or (ii) a compound of the general formula 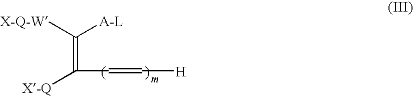
in which X, X′, 0, W′, A and L have the meanings given for the general formula II, and in addition if X represents a polymer, X′ and electron-withdrawing group W′ together with the interjacent atoms may form a ring, and m represents an integer 1 to 4; with a protein or a peptide containing at least two histidine residues; and if desired, reducing the electron-withdrawing group W′.

23. A process as claimed in claim 22, which is carried out at a pH in the range of from about 6.8 to about 8.5

24. A process as claimed in claim 23, which is carried out at a pH in the range of from about 7.0 to about 8.0.

25. A process as claimed in claim 24, which is carried out at a pH in the range of from about 7.5 to about 8.0.

26. A pharmaceutical composition comprising a compound as claimed in claim 1 together with a pharmaceutically acceptable carrier.

27. A compound as claimed in claim 1 or a pharmaceutical composition comprising the compound together with a pharmaceutically acceptable carrier for use in therapy.

28. A method of treating a patient which comprises administering a pharmaceutically effective amount of a compound as claimed in claim 1 or a pharmaceutical composition comprising the compound together with a pharmaceutically acceptable carrier to the patient.
